(19) United States
(12) Patent Application Publication
(10) Pub. No.: US 2011/0282168 A1
(43) Pub. Date: Nov. 17, 2011

(54) Health monitoring device and methods thereof

(75) Inventors: Alan Robert Weiss, Austin, TX (US); Samuel Burrill Fuller, Austin, TX (US); Oscar Paul Frowijn, Austin, TX (US); Afsheen David Gholami, Austin, TX (US); Gad Levy Krumholz, Austin, TX (US)

(73) Assignee: WALDO NETWORKS, Austin, TX (US)

(21) Appl. No.: 13/107,254

(22) Filed: May 13, 2011

Related U.S. Application Data
(60) Provisional application No. 61/334,857, filed on May 14, 2010.

(51) Int. Cl. A61B 5/1455; G06F 15/16; A61B 5/01; A61B 5/0476; A61B 5/021; A61B 5/0432

(52) U.S. Cl. 600/323; 600/485; 600/523; 600/549; 600/544; 709/219

(57) ABSTRACT

A health monitoring system includes a client device having one or more sensors to take medical or environmental measurements. The client device can interact with a server device to guide the user through the use of a sensor. The state of the sensors is controlled at the server to provide for flexibility of the health monitoring system.

### FIELD OF THE DISCLOSURE

This disclosure relates generally to electronic devices and more particularly to medical devices.

### BACKGROUND

As the cost of health care continues to increase, medical professionals, health care providers, and patients seek ways to control costs while providing a quality health care experience. Typically, in order to obtain medical care, patients have had to travel to a central location, such as a doctor's office or hospital. This can result in long wait times and is inefficient for the patient and the health care provider.

### BRIEF DESCRIPTION OF THE DRAWINGS

FIG. 1 illustrates a block diagram of a health monitoring system in accordance with one embodiment of the present disclosure.

FIG. 2 is a block diagram of a health monitoring device of FIG. 1 in accordance with one embodiment of the present disclosure.

FIG. 3 is a block diagram of a health monitoring server of FIG. 1 in accordance with an embodiment of the present disclosure.

FIG. 4 is a block diagram of the health monitoring device of FIG. 2 in accordance with one embodiment of the present disclosure.

FIG. 5 is a flow diagram of a method of controlling a health monitoring device in accordance with one embodiment of the present disclosure.

FIG. 6 is a block diagram of a health monitoring device of FIG. 1 in accordance with one embodiment of the present disclosure.

The use of the same reference symbols in different drawings indicates similar or identical items.

### DETAILED DESCRIPTION OF DRAWINGS

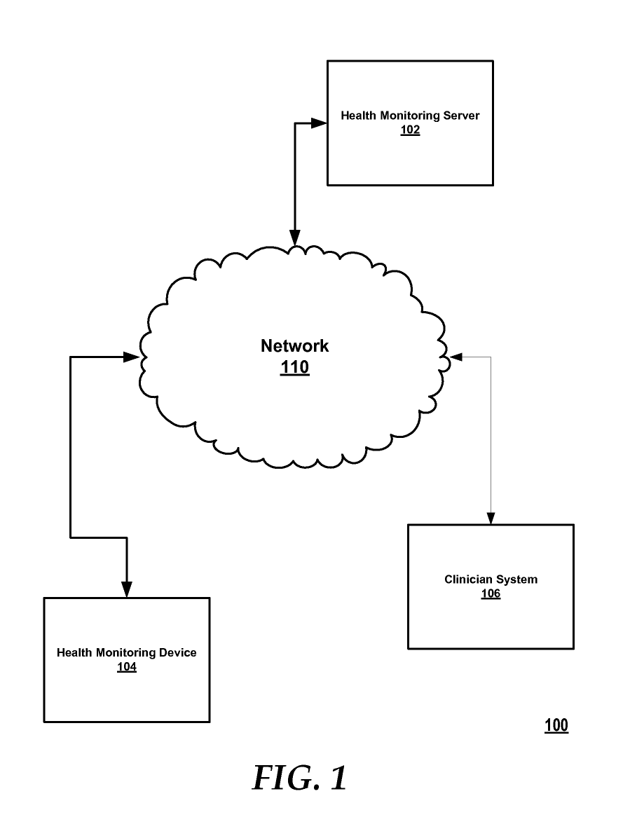

FIG. 1 illustrates a block diagram of a health monitoring system 100 in accordance with one embodiment of the present disclosure. Health monitoring system 100 includes a health monitoring server 102, a health monitoring device 104, and a clinician system 106, each connected to a network 110. The network 110 is a communications network operable to transfer information between connected devices. In an embodiment, the network 110 is a packet-switched wide-area network, such as the Internet, that provides a communication backbone for computer devices, including portable devices. It will be appreciated that network 110 can include one or more networks operated by different service providers. For example, network 110 can include one or more wide area networks operated by a communications service provider, and one or more local area networks, operated by a home or business user.

The health monitoring device 104 is a computer device operable to allow an individual user to determine and record medical information, such as diagnostic information, symptom information and the like. Accordingly, the health monitoring device 104 can include one or more sensors to facilitate implementing medical procedures to determine designated medical information. For example, the health monitoring device 104 can include a blood pressure pad, an electronic stethoscope, an examination camera, a weight scale, a balance sensor, an electrocardiograph sensor, a spirometer/peak flow sensor, a thermometer or other temperature sensor, an examination camera, an electroencephalography, an emergency response button, an ambient activity sensor and the like. The sensors can also include output devices that provide user notifications, such as lights or other illumination devices. For example, the health monitoring device 104 can include one or more lights that can be illuminated to indicate to the user that a medical procedure, such as recording of blood pressure is in progress, or has been completed. The health monitoring device 104 can also include a display device, such as a touch screen, to allow the user to interact with device, including changing device settings, viewing instructional videos, conducting a video chat with a health care provider, and the like. In an embodiment, the health monitoring device 104 is a portable device that a user can easily move to perform medical procedures, such as determination of a pulse, blood pressure, and the like.

Health monitoring server 102 is a computer device that executes one or more programs to facilitate the determination and recording of designated medical information. As used herein, a program is a set of instructions to manipulate a processor to perform one or more specified functions. In particular, and as described further herein, health monitoring server 102 can execute one or more programs to control the operation of remote health monitoring devices, such as health monitoring device 104. The health monitoring server 102 also can include web server programs to provide web pages, both to home users of health monitoring devices, and to medical professionals that can be used as user interfaces. The web pages provide a flexible interface for both the home user, such as a patient, and medical professional to communicate with the health monitoring server 102. In addition, the health monitoring server can interact with one or more databases (not shown) to create, record, and update electronic health records (EHRs) in response to interactions with home users and medical professionals.

Clinician system 106 is a computer system employed by a clinician or other medical professional to communicate with patients. Accordingly, the clinician system 106 executes one or more programs, such as a web browser, medical procedure program, chat program, video interface program, and the like, to allow a medical professional to interact with a patient located remotely from the professional.

In operation, the health monitoring server 102 interacts, via the network 110, with both the health monitoring device 104 and the clinician system 106, to facilitate implementing a medial procedure program that can determine and record medical information. This can be better understood with reference to an example where health monitoring server 102 facilitates the assessment of a user's blood pressure. In particular, the health monitoring server 102 determines that the blood pressure of the user associated with health monitoring device 104 is to be assessed. This determination can be based on the user's request, based on a schedule indicated by a medical professional, and the like.

In a specific embodiment of the present disclosure, the health monitoring server notifies a patient when it is time for the patient to determine the blood pressure. For example, the health monitoring server 102 can communicate information to the health monitoring device 104 to indicate that the blood pressure is to be taken. This information can be provided as a web page and displayed via the browser program at the health monitoring device 104. In response, the user can interact with the web page to indicate that she is ready to take the blood pressure measurement. The health monitoring device 104 communicates the user's readiness to the health monitoring server 102, which can set the state of one or more sensors at health monitoring device 104 to begin the measurement process. For example, the health monitoring server 102 can illuminate one or more lights or other visual indicators of a blood pressure cuff attached to the health monitoring device 104. These visual indicators can inform the user that the blood pressure cuff is the proper device to take the blood pressure measurement. The visual indicators can provide further information, such as indicate locations on the blood pressure cuff to identify how the cuff should be placed on the user's body to take the measurement.

In addition, the health monitoring server 102 can provide additional information to the user to facilitate taking of the blood pressure measurement. For example, via the web server program the health monitoring server 102 can provide graphical or textual instructions to the user as to how the blood pressure reading should be taken. Thus, the health monitoring server 102 can provide streaming video, a series of slides, audio information, and the like, to instruct the user how to connect a blood pressure pad to the health monitoring device 104, how to affix the pad to the body so that an accurate blood pressure reading can be taken, and the like. In an embodiment, the health monitoring server 102 can facilitate communication between a health provider using clinician system 106 and the user of health monitoring device 104. For example, the health monitoring server 102 can provide video chat services between the clinician system 106 and the health monitoring device 104, so that a health professional can guide the user through the blood pressure measurement.

Once the blood pressure measurement has been taken, the health monitoring device 104 can communicate the measurement to the health monitoring server 102. In response, the health monitoring server 102 can update one or more records, such as an EHR. In addition, the health monitoring server 102 can notify the clinician system 106 of the measurement. In an embodiment, the health monitoring server 102 can communicate the measurement in response to determining the measurement exceeds a designated threshold. For example, if the blood pressure measurement exceeds a threshold indicating a potential health problem, the health monitoring server 102 can provide a notification to the clinician system 106, so that appropriate action can be taken.

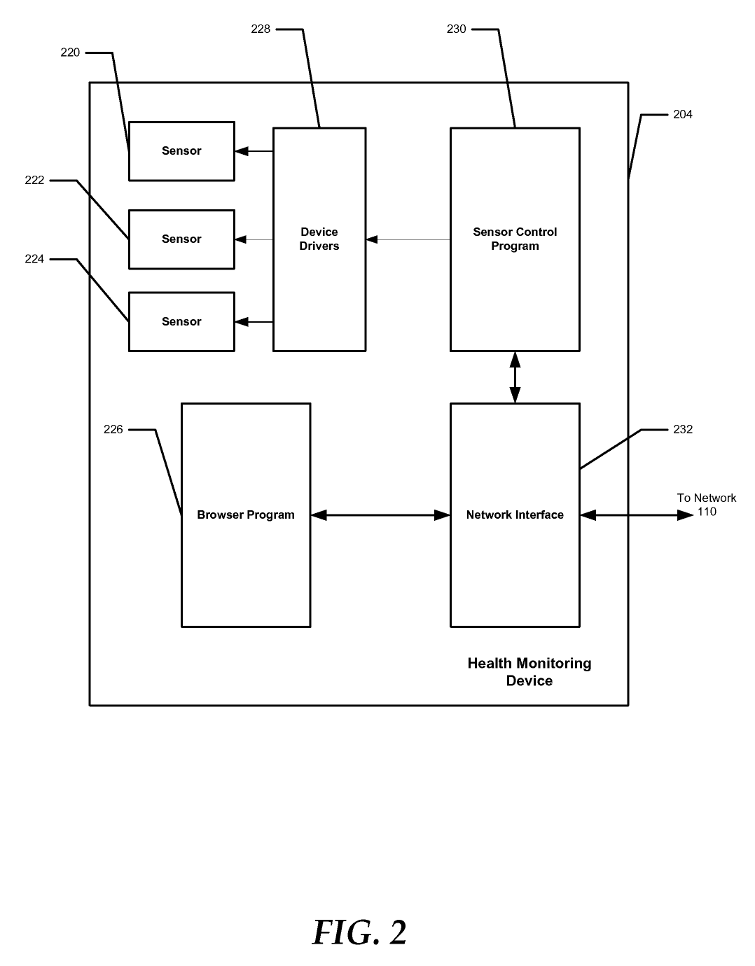

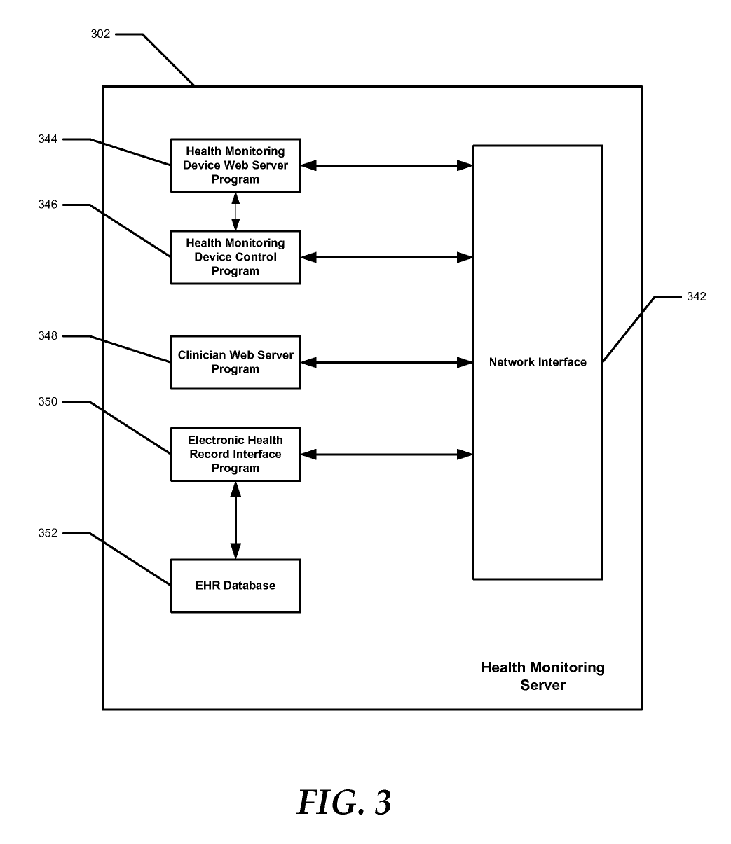

In an embodiment, the health monitoring server 102 controls the state of each sensor of the health monitoring device 104. This can simplify the design and operation of health monitoring device 104, and allow simplified updating of health monitoring control and other programs at the health monitoring server 102. This can be better understood with reference to FIGS. 2 and 3.

FIG. 2 illustrates a block diagram of a health monitoring device 204, corresponding to a particular embodiment of the health monitoring device 104 of FIG. 1. The health monitoring device 204 includes input/output devices/sensors 220, 222, and 224 (hereinafter referred to as sensors 220, 222, and 224), a browser program 226, device drivers 228, a sensor control program 230, and a network interface 232.

Each of the sensors 220, 222, and 224 is a device operable to provide information to the user, measure medical or environmental information about the user, receive information from the user, and any combination thereof. Accordingly, the sensors 220, 222, and 224, can be one or more of a light emitting diode (LED) or other light source, an audio source, audio sensor, such as a microphone, a camera, a digital stethoscope, a blood pressure pad, pressure sensor, blood oxygen sensor, magnifying device, weight scale, thermometer or other temperature sensor, balance sensing device, peak flow device, spirometer device or other device that senses lung capacity, otoscope, odor detection device and the like. For purposes of discussion, the sensors 220, 222, and 224 will be referred to as sensors, but it will be appreciated that as used herein a sensor can be a device that only provides information to the user, such as an LED or other light source, audio source, and the like. For medical information measurement devices, such as the blood pressure pad or digital stethoscope, the sensor is operable to translate a designated type of physical information, such as a pressure, which can used to determine blood pressure or a pulse rate, to one or more digital values that can be communicated and stored by a computer device. In an embodiment, one or more of the sensors 220-224 is a physically detachable sensor that can be attached to the health monitoring device 204 via an interface port, such as a universal serial bus (USB) connection or other port. Further, one or more of the sensors 220-224 can communicate with the health monitoring device wirelessly, via a wireless connection that complies with a BLUETOOTH protocol, 802.11 protocol, ANT, ANT+, Zigbee, or other wireless protocol.

The device drivers 228 are software programs operable to provide an interface for an associated sensor device. Accordingly, the device drivers 228 are operable to receive commands to control a designated sensor, translate the commands into a designated format, such as a format that conforms to a designated standardized protocol associated with the sensor, and communicate the translated command to the sensor. In addition, the device drivers 228 can receive information from each of the sensors, translate the information into a customized or standard protocol, and communicate the translated information to another program. The device drivers 228 thus provide a software interface to control one or more sensors.

The sensor control program 230 is a program operable to control one or more sensors according to received instructions. Thus, based on a received command, the sensor control program 230 identifies one or more sensors associated with the instruction, and communicates commands to the identified sensors in order to execute the command. For example, the sensor control program 230 can receive an instruction indicating that a light should be illuminated. The sensor control program 230 identifies the indicated sensor associated with the light, and communicates a command to illuminate the light.

The browser program 226 is a program operable to receive web page information in hyper text markup language (HTML) or other format, and display the web page based on the received information. In addition, the browser program 226 can receive information via a user interface, such as a touch screen (not shown) and communicate the received information to indicate how the user is interacting with the web page.

The network interface 232 is a device operable to provide a physical layer interface to the network 110. Accordingly, the network interface 232 can receive information from one or programs executing at the health monitoring device 204 and communicate that information to the network 110 for routing to a destination indicated by the one or more programs. In addition, the network interface 232 can receive information from the network 110 and route the received information to one or more executing programs.

In operation, the network interface 232 receives web page information from the health monitoring server 102, and communicates the information to the browser program 226. In response, the browser program 226 displays the web page associated with the information. As the user interacts with the displayed web page, the browser program communicates the interactions to the health monitoring server 102. Based on the interactions, the health monitoring server 102 communicates sensor control instructions to the network interface 232, which provides the sensor control instructions to the sensor control program 230 to control the state of one or more of the sensors 220-224. It will be appreciated that in some embodiments an operating system (not shown) may be executed by the health monitoring device, and the operating system mediates communications between the network interface 234 and the sensor control program 230. The operating system can also mediate communications between the network interface 234 and the browser program 226.

The network interface 234 communicates the instructions received from the health monitoring server 102 to the sensor control program 230, which controls the sensors, via the device drivers 228, to set the indicated sensors to the state indicated by the instructions. In addition, the sensor control program can receive information from the sensors 220-224, including state information and medical measurement information, and communicate the information to the health monitoring server 102. By controlling the web page displayed at the browser program 226 and the state of the sensors 220-224, the health monitoring server 102 can control health monitoring device 204 to determine medical information about the user.

This can be illustrated with reference to an example, where sensor 220 is a blood pressure pad and the user wishes to take a blood pressure measurement. Health monitoring server 102 communicates web page information for display via browser program 226. The web page information provides for a selection option “Take Blood Pressure.” Via touch screen or other input device (not shown), the user selects this option. The browser program 226 communicates the selection to the health monitoring server 102. In response, a management program at the server 102 determines that a blood pressure reading is to be taken. Accordingly, the health monitoring server 102 communicates web page information for display via browser program 226, instructing the user to attach the blood pressure pad (sensor 220) to the health monitoring device 204 (such as via a USB connection) and to place the blood pressure pad in an activated state, such as by depressing a button on the pad. The web page information can provide these instructions in a variety of ways, such as text, audio instruction, video instruction, via a video chat where a medical professional or technician guides the user, and the like.

In addition, the health monitoring server 102 can communicate instructions to the sensor control program 230 to change the state of sensor 220, so that the button to be pressed by the user to activate the pad is illuminated. The button can be on the blood pressure device itself or on the health monitoring device 204. The health monitoring server thus provides a visual cue to the user as to how the blood pressure pad should be activated. Once the user has activated the pad by pressing the button associated with sensor 220, the sensor control program 230 will communicate the state of the sensor 220 to the health monitoring server 102. In response, the health monitoring server 102 can change the provided web page information to instruct the user as to the next stage of the blood pressure measurement procedure.

The health monitoring server 102 controls the state of the sensors 220-224 through the measurement procedure to ensure the measurement is taken properly. Thus, for example, the health monitoring server 102 can set the state of lights or other visual indicators to assist the user in the measurement process. To illustrate, in response to the user actuates the button to activate the blood pressure cuff, the health monitoring server 102 can control the state of the sensors 220-224 to activate a set of lights on the blood pressure pad. The lights can be at different locations on the pad, with each light having a different color. In addition, the health monitoring server 102 can provide web page information to be displayed via the browser program 226 to instruct the user to place the pad such that the lights are oriented in a particular way or placed on designated locations on the user's body. In this way, the health monitoring server 102 can accurately instruct the user how to place the blood pressure pad in order to take a measurement. The health monitoring server 102 can also change the state of the sensors in order to effect a measurement. For example, once the user has indicated via the browser program 226 that the blood pressure pad is in place for measurement, the health monitoring server 102 can set the state of the pad to apply or relieve pressure at the pad as appropriate to take the measurement. The health monitoring server 102 can thus change the state of one or more of the sensors 220-224 during a measurement in order to effectuate the measurement.

The sensors 220-224 can also communicate information to the health monitoring server 102 during the measurement, which in response changes the state of the sensors. Thus, during an initial phase of a blood pressure measurement, the health monitoring server 102 can set the state of the blood pressure pad so that the pad is increasing pressure on the user. In response, the blood pressure pad can communicate pressure information to the health monitoring server 102. Once the pressure information has reached a designated threshold, the health monitoring server 102 can change the state of the pad such that the pad is releasing, rather than increasing pressure. The blood pressure pad communicates pressure readings during both the increasing-pressure and decreasing-pressure phases to the health monitoring server 102. Based on this information, the health monitoring server 102 can determine a blood pressure measurement for the user.

In addition, the sensors 220-224 provide state information to the health monitoring server 102 so that the server can determine if the measurement is being taken properly. Thus, for example, position sensors can be incorporated into the blood pressure pad, and the state of these sensors can indicate whether the pad has been properly affixed to the user for measurement.

In another embodiment, the health monitoring device 204 can temporarily store the results of a medical measurement in response to determining the results cannot be communicated to the health monitoring server 102. For example, the health monitoring device 204 can be placed in an offline mode, such that the device does not communicate with the health monitoring server 102 while in the offline mode. After the health monitoring device 204 has been placed in an online mode, such that it can communicate with the health monitoring server 102, the server can request any medical measurement results stored at the device 204. In response, the health monitoring device 204 communicates the stored medical measurements. The health monitoring device 204 can also store a program to provide a version of the user interface to the user, so that measurements can be taken when the network is not available.

The state of the sensors 220-224 can be set and adjusted by the health monitoring server to take a variety of medical measurements, depending on the type of sensors 220-224. For example, if one of the sensors 220-224 is a camera device used to perform remote medical examinations, the health monitoring server 102 can set the state of the sensors 220-224 to adjust parameters of the camera, such as magnification, illumination, and other factors. The state can be set automatically by the health monitoring server 102 based on predefined measurement programs, or set based on requests by a medical professional, such as a professional using the clinician system 106. Thus, for example the medical professional can interact with the remote user via the browser program 226, such as via video chat. During this interaction, the medical professional can request the user to activate the camera device and place the camera so that the medical professional can view a portion of the user's body. The camera communicates images to the health monitoring server 102, which in turn provides those images to the medical professional via the clinician system 106. In response to viewing the images, the medical professional can request changes to the state of the camera, such as an increase in magnification, illumination, or the like. In response, the health monitoring server 102 sets the state of the camera in accordance with the professional's request. Thus, the professional can conduct a remote examination of the user via the health monitoring server 102.

In the illustrated embodiment, the state of the sensors 220-224 is controlled by instructions from the health monitoring server 102, rather than instructions generated by the browser program 226. In other words, although the sensor control program 230 can set the state of a designated sensor, it does so only in response to instructions from the health monitoring server 102, and not in response to instructions from the browser program 226. Thus, the state of each of the sensors is not determined by the browser program 226, but rather by one or more programs executing at the health monitoring server 102. This simplifies the design of the health monitoring device 204, reducing the need for the device to store and execute complex medical analysis programs. In addition, the health monitoring server 102 can interact with multiple remote health monitoring devices. Accordingly, any change in the programs that manage the state of the sensors, such as the programs that control the measurement of medical information, is deployed to each remote device by updating the programs at the health monitoring server 102. In other words, the programs can be updated without having to update each local health monitoring device.

In an embodiment, communications between the health monitoring server 102 and the health monitoring device 204, including communications indicative of medical measurements, are encrypted to improve patient security and privacy. Further, to provide patient security and privacy, in one embodiment the health monitoring device 204 does not store or retain medical measurement information after the information has been communicated to the health monitoring server 102. Thus, if the health monitoring device 204 is lost or stolen, the user's medical information cannot be accessed. In addition, because all medical measurements are controlled at the health monitoring server 102, the server can decline to take any measurements at a health monitoring device that has been identified as lost or stolen.

Further, the health monitoring server 102 can communicate customized information to each of a plurality of health monitoring devices to provide different information to different users. For example, how a medical measurement should be taken can vary depending on factors that vary between individuals, such as the underlying medical condition, age, and other physical parameters associated with each individual. Accordingly, for each medical measurement, the health monitoring server 102 can control the state of the sensors at the health monitoring device to provide a customized experience for a particular user, based on that user's physical parameters. In addition, because the state of the sensors are controlled at the health monitoring server 102, the customized information provided for each user can be more easily adjusted, as the need for complex device patches or other updates is reduced.

FIG. 3 illustrates a health monitoring server 302, corresponding to a particular embodiment of the health monitoring server 102 of FIG. 1. The health monitoring server 302 includes a network interface 342, a health monitoring device web server program 344, a health monitoring device control program 346, a clinician web server program 348, an EHR interface program 350, and an EHR database 352.

The network interface 342 is a device operable to provide a physical layer interface to the network 110. Accordingly, the network interface 232 can receive information from one or programs executing at the health server device 302 and communicate that information to the network 110 for routing to a destination indicated by the one or more programs. In addition, the network interface 342 can receive information from the network 110 and route the received information to one or more executing programs.

The health monitoring device web server program 344 is a program that provides web page information to a health monitoring device, such as health monitoring device 204. Health monitoring device control program 346 is a program operable to control the state of the health monitoring device 204 based on the user's interactions with the displayed web page. The programs 344 and 346 thus operate together to control the health monitoring device 204 to allow the user to determine medical information, such as measurement of vital signs, as described above with respect to FIG. 2. In particular, health monitoring device control program 346 determines the state of each sensor at the remote health monitoring device 204, and also receives information from the health monitoring device web server program 344 to determine how the user is interacting with a displayed web page. Based on this information, the health monitoring device control program 346 sets the state of each sensor. Thus, for example, as the user employs a particular sensor to take a measurement, the health monitoring device control program 346 set the state of sensors at the device, both to guide the user in taking the measurement, as well as control the measurement parameters.

In an embodiment, the health monitoring device control program 346 can authenticate received user information to determine the user of a particular health measurement device. The authentication information can be password information, device identification information, and the like. Based on the authentication information, the health monitoring device control program 346 can identify the user of the health monitoring device. Based on the identified user, the health monitoring device control program 346 can control sensors at the remote device both to guide the user in taking the measurement in a fashion customized for the identified user, as well as control the measurement parameters in a fashion customized for a particular user. For example, the health monitoring device control program 346 can determine, based on identifying a first user based on the authentication information, that the first user is a diabetic having a particular type of insulin measurement device attached as one of the sensors for the associated health monitoring device. Accordingly, the health monitoring device control program 346 can control the sensors at the remote device to guide the user in taking an insulin measurement in a fashion customized for the particular type of insulin measurement device associated with the first user. For a second user having a different type of insulin measurement device, the health monitoring device control program 346 can control the sensors at the remote device to guide the second user in taking an insulin measurement in a fashion customized for the different type of insulin measurement device associated with the second user. It will be appreciated that, because the determination of the user, and associated sensor control, is done at the health monitoring server 102, the health monitoring devices need not employ a customized patch or other customized adjustment in order to provide a customized experience for each user.

Clinician web server program 348 is a program to provide web page information to clinician system 106. Via the provided web pages, the clinician web server program 348 can communicate medical information, such as measurements taken by health monitoring device 104, to a medical professional at clinician system 106. In addition, the clinician web server program 348, in conjunction with health monitoring device web server program 344, can provide an interface for the medical professional and the user to communicate. For example, the programs can provide a web chat or video chat interface, thereby allowing the medical professional to guide the user in taking a particular measurement, to suggest a course of treatment, adjust a medical plan, and the like.

EHR interface program 350 provides an interface to EHR database 352. In particular, EHR interface program 350 is operable to automatically update EHR database 352 based on vital sign or other measurements taken by the user at health monitoring device 104.

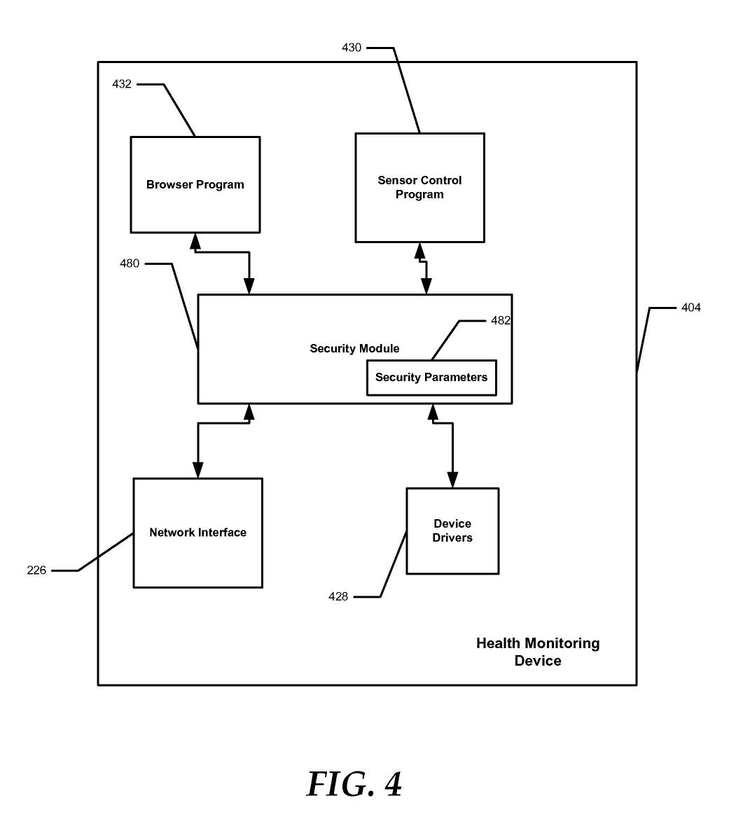

FIG. 4 illustrates a block diagram of a health monitoring device 404, corresponding to a particular embodiment of the health monitoring device 204 of FIG. 2. The health monitoring device 404 includes a browser program 426, device drivers 428, a sensor control program 430, and a network interface 432, each configured similarly as the corresponding modules of health monitoring device 204 of FIG. 2. Thus, for example, device drivers 428 are configured to provide an interface for one or more sensor devices (not illustrated at FIG. 4).

Health monitoring device 404 also includes a security module 480 to provide a gate keeping function for communications between other modules of the device. In particular, the security module 480 can be a hardware module or a software module, such as an operating system, or a combination thereof, that monitors communications from each of the browser program 426, the device drivers 428, the sensor control program 420, and the network interface 432 and determines, based on security parameters 482, whether each communication can be provided to the target module. To illustrate, the security module 480 can receive a communication from a first module, such as the browser program 426, targeted to a second module, such as the device drivers 428. The security module 480 can determine, based on the security parameters 482, whether the browser program 426 has permission to communicate with the device drivers 428. If so, the security module 480 allows the communication to be provided to the device drivers 428. If the browser program 426 does not have permission, the security module 480 prevents the communication from being provided to the device drivers 428 and communicates an error message to the browser program 426. In another embodiment, the device drivers 428 can bypass the security module 480.

In an embodiment, the security parameters 482 are set so that the browser program 426 is sandboxed from the device drivers 428 and the sensor control program 420. That is, the browser program 426 is restricted so that it cannot communicate with these modules, except by communicating through the health monitoring server 102 as described. Thus, the security module 480 isolates the browser program 426 such that any interactions between the browser and the device drivers 428 are mediated by the health monitoring server 102.

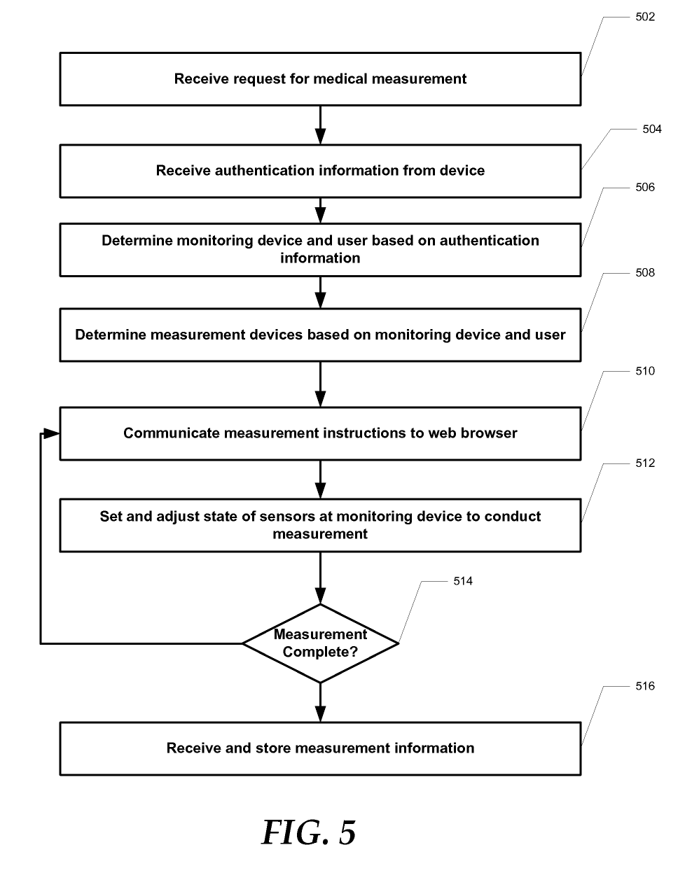

FIG. 5 is a flow diagram of a method of controlling a health monitoring device in accordance with one embodiment of the present disclosure. At block 502, the health monitoring server 102 receives a request from the health monitoring device 104 to take a particular measurement. This request can be received based on a user's interaction with the health monitoring device 104, based on a periodic or scheduled time to take the measurement or the like. At block 504, the health monitoring server 102 receives authentication information from the health monitoring device 104. The authentication information can be information that identifies the device itself, the device user, or any combination thereof. Accordingly, the authentication information can be password or other security information (such as a private key) associated with a particular user, device identification information, such as a serial number or private key, that identifies the health monitoring device 104, or any combination thereof.

At block 506, the health monitoring server 102 identifies the health monitoring device 104 and the user thereof based on the authentication information. In an embodiment, the authentication information identifies a device profile associated with the health monitoring device 104 and stored at the health monitoring server 102. The device profile can indicate a device type associated with the device 104 (such as a device revision number or other device type) and indicate the sensors associated with the device. For example, the device profile can indicate the particular medical measurement devices associated with the health monitoring device 104. For example, the device profile can indicate whether the health monitoring device 104 includes (or is connected to) a blood pressure pad, camera, stethoscope, or other medical devices. In an embodiment, the device profile is customized for each user, device, or any combination thereof. Thus, for example, a device profile associated with a particular medical measurement device can indicate that the device includes a blood pressure pad and camera, but does not include a stethoscope, while the device profile associated with a different medical measurement device includes a stethoscope and a blood oxygen measuring device, but does not include a camera. Based on the device profile, the health monitoring server 102 can provide customized information for each user and medical measurement device. Thus, for example, if the device profile indicates a medical measurement device does not include a particular type of medical measurement device, the health monitoring server 102 will not provide any option or other information to the user of that health monitoring device to take a measurement with the omitted device.

Further, the health monitoring server 102 can tailor state information for the medical measurement devices based on the measurement devices indicated by the device profile. For example, the device profile information may indicate that a health monitoring device is connected to a designated one of two possible types of examination cameras. The two types of examination cameras can have different options, sensors, and other features. Accordingly, each camera type may require different state information to be communicated during a medical measurement. Accordingly, based on the device profile, the health monitoring server 102 can determine the type of examination camera connected to the health monitoring device 104 and therefore communicate the appropriate state information to the camera during a medical measurement.

At block 508, the health monitoring server 102 determines medical measurement devices associated with the user of the health monitoring device 104. In an embodiment, this determination is made based on the device profile information described above. Based on the requested medical measurement, as well as the determined measurement devices, the health monitoring server 102 communicates at least a portion of the instructions to take the measurement to the browser program 226. The instructions can include video clips or streaming video information, slides, pictorial or textual information, audio information, or any combination thereof. At block 512, the health monitoring server communicates with the sensor control program 230 to set and adjust the state of the sensors at the medical measurement device taking the medical measurement. In an embodiment, the state of the sensors can be set and adjusted based on requests from a medical professional that is remotely monitoring the medical measurement at the clinician system 106.

At block 514, the health monitoring server 102 determines whether the medical measurement is complete. In an embodiment, the health monitoring server 102 receives feedback from the sensors at the medical measurement device during the measurement and, based on the feedback, determines whether the medical measurement is complete. In another embodiment, the health monitoring server 102 determines whether the medical measurement is complete based on instructions received from a medical professional that is remotely monitoring the medical measurement at the clinician system 106. If the medical measurement is not complete, the method flow returns to block 510, and the health monitoring server continues to provide instructions and set and adjust the state of the sensors at the health monitoring device 104 to effectuate the medical measurement. Once the medical measurement is complete, the health monitoring server receives and stores information based on the medical measurement. The information can be a blood pressure reading, blood oxygen reading, recorded visual image, or any other medical information. In an embodiment, the information is stored at an electronic health record associated with the user of the health monitoring device 104. The information can also be provided to a medical professional at the clinician system 106 or other location, where it can be displayed to the clinician or other medical practitioner.

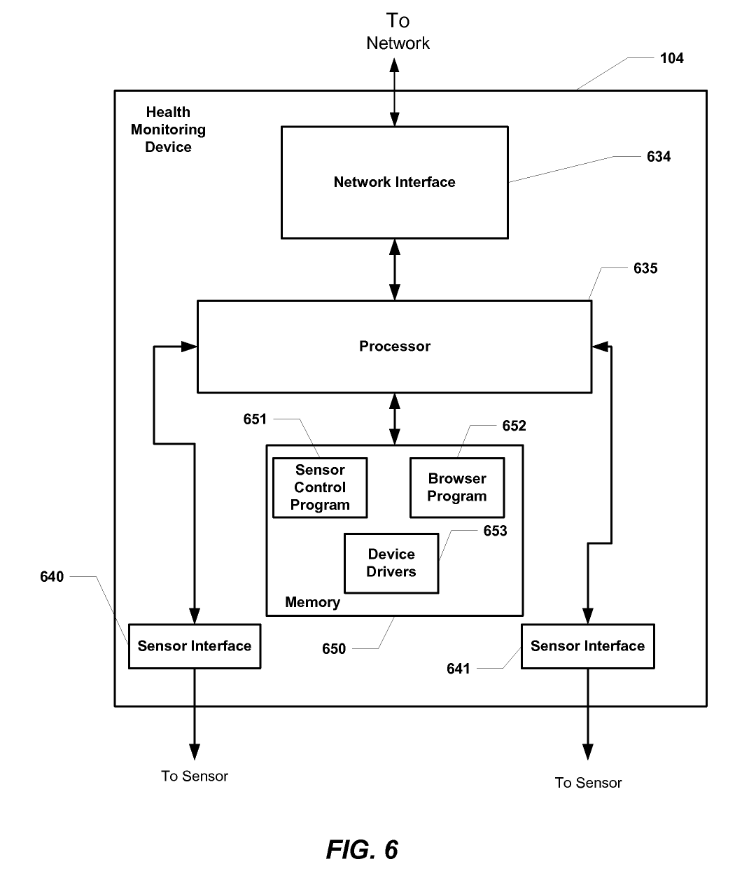

FIG. 6 illustrates a block diagram of the health monitoring device 104 in accordance with one embodiment of the present disclosure. As illustrated, the health monitoring device 104 includes a network interface 634, a processor 635, sensor interfaces 640 and 641, and a memory 650. The processor 835 is connected to the network interfaces 634, to the memory 650, and to each of the sensor interfaces 640 and 641.

The network interface 634 is a device configured to provide a physical and logical layer interface to a network so that the health monitoring device can communicate with the health monitoring server 102. Thus, the network interface 634 can be a network interface card (NIC) or other device that communicates with the health monitoring server via a wide area network, local area network, or any combination thereof.

Each of the sensor interfaces 640 and 641 are configured to provide a communications interface to one or more sensor devices via one or more physical connection associated with the interface. Accordingly, each of the sensor interfaces 640 and 641 can be a wired or wireless network interface, such as a network interface card (NIC), a USB interface, an RS-232 interface, a Bluetooth interface, and the like. In an embodiment, each of the communications interfaces 640 and 641 is a different kind of interface. For example, communication interface 640 can be a wireless interface configured to communicate with a sensor wirelessly while sensor interface 641 is a USB interface.

The processor 635 is a general purpose or application-specific processor configured to execute sets of instructions in order to perform tasks associated with the instructions. Although processor 635 is illustrated as a single processor, the processor 635 can represent multiple processors, a single processor having multiple processor cores, or any combination thereof.

The memory 650 is a computer readable medium configured to store information and retrieve stored information based on received commands. Accordingly, the memory 650 can be volatile memory, non-volatile memory, or any combination thereof. For example, memory 650 can be random access memory (RAM), read only memory (ROM), flash memory, a hard disc drive, solid state memory, or any combination thereof. The memory 650 stores programs of instructions for execution at a processor, including sensor control program 651 and browser program 652. In addition, memory 850 stores device drivers 653, which are programs that control the processor 635 to interface with one or more sensors, such as a blood pressure measuring device, stethoscope, medical examination camera, and the like via the sensor interfaces 640 and 641.

In operation, the processor 635 accesses the memory 650 to execute one or more of the stored programs. During execution of the programs, the processor controls and interfaces with the network interface 634 and the communication interfaces 640 and 641 to perform one or more of the methods described herein. For example, during execution of the sensor control program 651 and the browser program 652, the processor 635 can take a medical measurement by controlling the state of the sensors based on communications received from the health monitoring server 102.

Other embodiments, uses, and advantages of the disclosure will be apparent to those skilled in the art from consideration of the specification and practice of the disclosure disclosed herein. For example, it will be appreciated that although the health monitoring server 102 is illustrated as a single device, in other embodiments, the functions of the health monitoring server 102 can be performed by multiple server devices. Further, each server device can be configured to perform different functions. Thus, for example, one server can be configured to store the database of electronic health records, while another server is configured to execute the health monitoring device control program 346, and still another server executes the health monitoring device web server program 344. Similarly, the clinician system 106 can include one or more servers, clients, or other computer devices to perform the functions of the system. Accordingly, the specification and drawings should be considered exemplary only.

What is claimed is:

1. A method, comprising:
communicating via a network, from a server device to a first program at a remote device, first information to be displayed by the first program;
receiving at the server device via the network second information associated with a request to take a medical measurement at the remote device;
determining at the server device, first state information based on the second information; and
communicating the first state information to a second program at the remote device to set the state of a sensor at the remote device, the second program separate from the first program.

2. The method of claim 1, wherein the first state information is customized for a first user.

3. The method of claim 1, wherein the first state information is customized for the first device.

4. The method of claim 1, further comprising:
receiving authentication information from the remote device; and
determining the first state information based on the authentication information.

5. The method of claim 3, wherein determining the first state information comprises determining a sensor type associated with a first user based on the authentication information and determining the state information based on the sensor type.

6. The method of claim 4, wherein determining the sensor type comprises determining a type of medical measurement device.

7. The method of claim 1, wherein the sensor comprises a stethoscope.

8. The method of claim 1, wherein the sensor comprises a blood pressure measurement device.

9. The method of claim 1, wherein the sensor comprises a blood oxygen measurement device.

10. The method of claim 1, further comprising providing a stream of video information from the server device to the remote device.

11. The method of claim 10, wherein providing the stream of video information comprises providing a video chat session to the remote device.

12. A device, comprising:
a communication interface to communicate with a remote server via a network;
a sensor interface to communicate with a sensor device;
a processor coupled to the communication interface and the sensor interface; and
a computer readable medium coupled to the processor to store a first program and a second program, the first program comprising a plurality of instructions to manipulate the processor to display first information received via the communication interface, and a second program comprising a plurality of instructions to manipulate the processor to:
receive from the server device, first state information in response to a request to take a medical measurement; and
control a state of the sensor device based on the first state information.

13. The device of claim 12, wherein the first state information is customized for a first user.

14. The device of claim 12, wherein the first state information is customized for the first device.

15. The device of claim 12, further comprising:
wherein the second program comprises instructions to:
provide authentication information in response to a request received from the server; and
receive the first state information in response to the authentication information.

16. The device of claim 12, wherein the sensor comprises a stethoscope.

17. The device of claim 12, wherein the sensor comprises a blood pressure measurement device.

18. The device of claim 12, wherein the sensor comprises a blood oxygen measurement device.

19. The device of claim 12, wherein the sensor comprises a weight scale.

20. The device of claim 12, wherein the sensor is selected from the group consisting of: a balance sensor, an electrocardiograph sensor, a spirometer/peak flow sensor, a thermometer or other temperature sensor, an examination camera, an electroencephalography, an emergency response button, and an ambient activity sensor.
